(19) United States
(12) Patent Application Publication
(10) Pub. No.: US 2017/0135678 A1
(43) Pub. Date: May 18, 2017

(54) RECOVERING ANALYTES BY REVERSE PLASMAPORATION

(71) Applicant: EP Technologies LLC, Akron, OH (US)

(72) Inventors: Sameer Kalghatgi, Copley, OH (US); Daphne Pappas Antonakas, Hudson, OH (US); Robert L. Gray, Kent, OH (US); Abhishek Juluri, Akron, OH (US)

(21) Appl. No.: 15/352,817

(22) Filed: Nov. 16, 2016

Related U.S. Application Data
(60) Provisional application No. 62/256,960, filed on Nov. 18, 2015.

(51) Int. Cl. A61B 10/00; A61M 5/172; A61B 5/00; A61B 5/145; A61B 5/15

(52) U.S. Cl. CPC A61B 10/0045; A61B 5/14532; A61B 5/14546; A61B 5/150099; A61B 5/4839; A61M 5/1723; A61M 2005/1726

(57) ABSTRACT

A subject's skin is exposed to non-thermal plasma, thereby enabling analytes in the interstitial fluid in the subject's body to migrate to the surface of the subject's skin. The concentration of analytes in the subject's blood can be determined based on the amount of these analytes.

### RELATED APPLICATIONS

The application claims priority to, and the benefits of, U.S. Provisional Patent Application Ser. No. 62/256,960 filed on Nov. 18, 2015 and titled RECOVERING ANALYTES BY REVERSE PLASMAPORATION, which is incorporated herein by reference in its entirety.

### BACKGROUND

A common way for diabetic patients to measure their blood glucose levels is by pricking a finger multiple times a day to recover a small sample of blood and then testing the sample for glucose concentration. Such invasive methods are disagreeable, not only because they are painful but also because of the inherent risk of infection due to human skin being penetrated. Repeated pricking of the finger also leads to undesirable side effects including scarring of the tissue and unreliable extraction of blood. In addition, the sharps used for this purpose represent a biohazardous waste disposal problem and pose a risk of illicit reuse.

Other less painful and less invasive approaches for measuring blood glucose levels, as well as the concentration of other analytes in a subject's blood, have also been proposed. Examples include implanting sensors, using lasers or miniature lancets to accomplish minimally invasive skin microporation, and non-invasive approaches such as using NIR spectroscopy, reverse iontophoresis, etc. However, none of these approaches has gained acceptance in routine clinical practice for a variety of reasons, including requiring long lead times, requiring large blood samples, and needing sophisticated, expensive equipment that is difficult to operate and error prone. In particular, one of the fundamental problems in noninvasive transdermal diagnostics is obtaining sufficient quantities of analyte for detection.

### SUMMARY

In accordance with this invention, reverse plasmaporation is used to recover analytes from below one or more layers of a subject's skin. The recovered analytes can then be analyzed to determine useful information about the patient's health.

Thus, this invention in one embodiment provides a process for recovering an analyte from inside a subject's body comprising exposing an area of the subject's skin to a non-thermal plasma, thereby enabling migration of the analyte from inside the subject's body to the surface of the subject's skin, and then recovering the analyte from this surface. The analyte so recovered can then be analyzed to determine useful information about the patient's health.

In another embodiment, this invention also provides a process for determining the concentration of an analyte in a subject's blood comprising exposing an area of the subject's skin to a non-thermal plasma, thereby enabling migration of the analyte from inside the subject's body to the surface of the subject's skin, and then determining the concentration of this analyte in the subject's blood based on the amount of analyte recovered from the surface of the subject's skin.

In still another embodiment, this invention provides a device for automatically maintaining the concentration of an analyte found in a subject's blood to within a desired predetermined range comprising a plasma generator arranged to expose the subject's skin to a non-thermal plasma thereby enabling migration of a quantity of this analyte to the surface of the subject's skin, an analytical device for measuring this quantity of analyte, a dispenser for delivering a quantity of this analyte or a drug that regulates this analyte to the subject, and a control system for directing the dispenser to administer a predetermined amount of this analyte or a drug that regulates this analyte to the subject in response to the amount of this analyte measured by the analytical device.

### BRIEF DESCRIPTION OF THE DRAWINGS

This invention may be more readily understood by reference to the following drawing wherein:

FIG. 1 schematically illustrates one type of device for exposing the skin of a subject to non-thermal plasma for facilitating recovery of analytes from the subject's body by reverse plasmaporation;

FIG. 2 schematically illustrates another type of device for exposing the skin of a subject to non-thermal plasma for facilitating recovery of analytes from the subject's body by reverse plasmaporation;

FIG. 3 schematically illustrates still another type of device for exposing the skin of a subject to non-thermal plasma for facilitating the recovery of analytes from the subject's body by reverse plasmaporation;

FIGS. 4(a), 4(b), 4(c) and 4(d) are schematic diagrams illustrating a comprehensive process carried out in accordance with exemplary embodiments in which reverse plasmaporation is used to recover analytes from inside a subject's body, after which the amounts of these recovered analytes are then used to determine the concentrations of these analytes in the subject's blood;

FIG. 5 is a schematic illustration of a vacuum device that can be used for applying suction to a subject's skin in accordance with certain embodiments of this invention; and

FIGS. 6-12 are graphs illustrating the results obtained in the working examples of this invention described below.

### DETAILED DESCRIPTION

### Plasma

Plasma is the fourth state of matter. Plasmas are fully (thermal) or partially ionized (non-thermal) gases, having one or more electrons that are not bound to an atom or molecule. Plasmas can operate far from thermodynamic equilibrium (temperature of gas is significantly lower than that of the highly energetic electrons) with high concentrations of energetic and chemically active species like reactive oxygen and nitrogen species (RONS), with gas temperature being essentially at room temperature.

Generally speaking, there are two types of plasmas, thermal or “hot” plasmas and non-thermal or “cold” plasmas. The primary difference between these two types of plasmas is the gas temperature of the plasma. In a thermal plasma, most of the molecules are ionized. As a result, the temperature of the plasma is typically a few thousand degrees Celsius or more. In such plasmas, the electrons, ions and neutrals are said to be in thermal equilibrium with one another in the sense that they exist at essentially the same high temperature. In contrast, in non-thermal plasmas, only a small percentage of the molecules are ionized. As a result, the ions and neutrals are at a much lower temperature than the electrons. The overall result is that the gas temperature of non-thermal plasma, as a whole, is often at or near room temperature.

In accordance with this invention, a non-thermal plasma is used to facilitate recovery of analytes from the interstitial fluid contained below one or more layers of a subject's skin. As well understood in the art, the interstitial fluid is the fluid found between the cells of the subject's skin and other tissue. For convenience, this process is referred to in this disclosure as “reverse plasmaporation.”

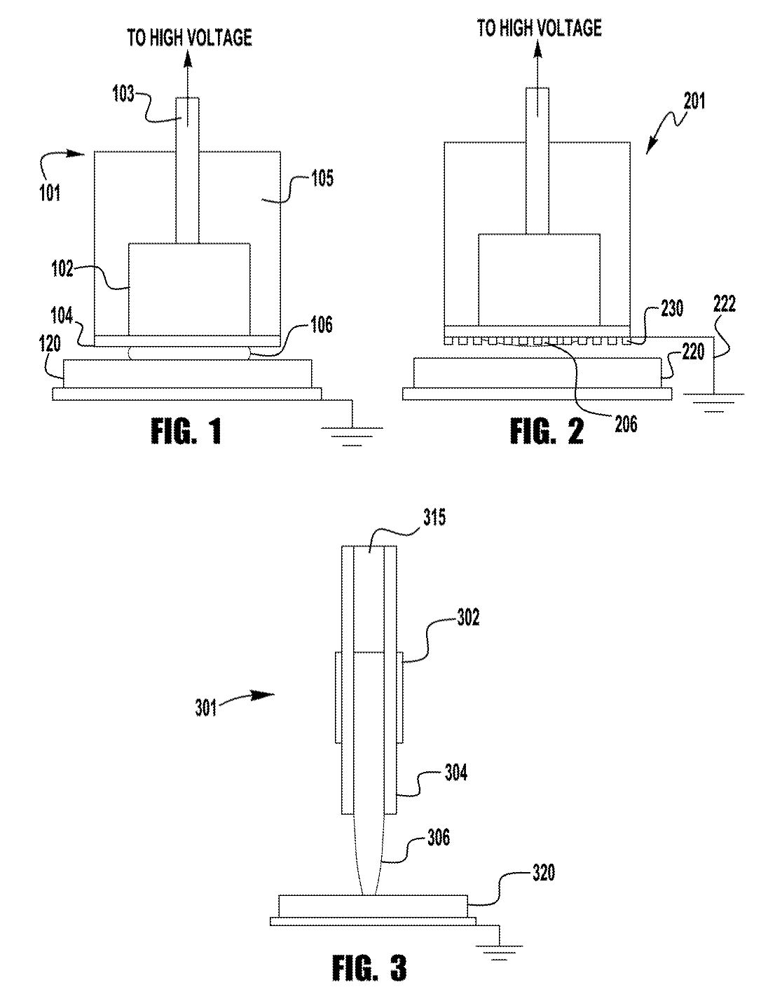

One known method for exposing the surface of an article to a non-thermal plasma is illustrated in FIG. 1, which shows a “large area” dielectric barrier discharge (“DBD”) plasma generating system 101 including high voltage source (not shown), conductor 103, housing 105, high voltage electrode 102 and dielectric barrier 104. In the particular embodiment shown, plasma generating system 101 is mounted a suitable distance, e.g., 2 mm, above the surface of target 120 to be treated, e.g. skin. Target 120 may be grounded, as shown in this figure, of it may be a floating ground, i.e., ungrounded.

During operation, the high voltage source is turned on and plasma 106, which forms between the dielectric barrier 104 and surface of target 120 and contains all three of electrons, ions and neutrals, treats the surface of target 120.

Another known method for exposing the surface of an article to a non-thermal plasma is illustrated in FIG. 2, which shows a plasma generating system 201 which is similar to plasma generating system 101 of FIG. 1, except that plasma generating system 201 further includes filter 230 in the form of a conductive mesh that is grounded by grounding conductor 222. Plasma generating system 201 operates in much the same way as plasma generating system 101, except that charged ions and electrons are prevented from passing through filter 230. The modified plasma 206 so obtained, which is typically referred to as an “afterglow” or “indirect” plasma, then irradiates target 220.

In this connection, for ease of description, in this disclosure, modified plasmas in which the charged ions and electrons have been removed such as produced in the system of the above FIG. 2 are referred to as “afterglow” plasmas. In contrast, plasmas containing all three of electrons, ions and neutrals such as produced by the system of FIG. 1 are referred to in this disclosure as “direct” plasmas. Meanwhile, unless otherwise clear from context, “plasma” will be understood to include both direct plasmas and afterglow plasmas.

Still another known method for exposing the surface of an article to non-thermal plasma is illustrated in FIG. 3. As shown there, a “jet-type” plasma generating system 301 includes a high voltage tubular electrode 302, a borosilicate glass tube 304 and a gas feed 315 for charging a suitable gas such as helium, argon, nitrogen, oxygen or mixtures thereof into the upper inlet end of glass tube 304. Plasma generating system 301 is also a floating-electrode dielectric barrier discharge (DBD) plasma generator, like plasma generating system 102 of FIG. 1. However, in the case of plasma generating system 301, the plasma is generated by the breakdown of the gas column between the high voltage tubular electrode 302 and the substrate 320. In this embodiment high voltage electrode 302 can be held farther away from the substrate 320 than the plasma generating system of FIG. 1. Additionally, in this embodiment we could treat a smaller focused area (3-5 mm) or treat a larger area by rastering the high voltage electrode 302 in a defined pattern over the surface of the substrate 320. Although DBD plasma generators have been shown and described herein, it is contemplated that other types of plasma generators may be used.

### Reverse Plasmaporation

In commonly assigned U.S. 2015/0094647 (35416/04025), the disclosure of which is incorporated herein by reference in its entirety, there is disclosed a non-invasive method for facilitating the transdermal delivery of drugs and other molecules from outside to inside a subject's body by exposing the subject's skin or tissue to a non-thermal plasma. As described there, the effect of this non-thermal plasma treatment, which is referred to there as “plasmaporation,” is to open pores is the subject's skin, thereby making it far more permeable to the passage of drugs and other molecules that are normally unable to diffuse through on their own. The overall result is that the rate at which particular drugs and other molecules can be delivered through the skin is greatly enhanced. In addition, the maximum size of molecules which can be delivered transdermally is also greatly increased. Indeed, molecules having molecular weights as high as 115,000 Da and higher can be delivered transdermally with this technology, which is far greater that than the maximum molecular weight for molecules delivered transdermally through regular, untreated skin, about 500 Daltons.

In accordance with this invention, essentially the same non-thermal plasma technology is also used to open pores in a subject's skin to make it more permeable, but in this invention this technology is used in reverse in the sense that it is used for facilitating withdrawal of analytes from the interstitial fluid found inside the subject's body to outside the subject's body transcutaneously rather than for delivering drugs and other molecules from outside to inside the subject's body. Thus, the transcutaneous movement occurring in this invention is referred to as “reverse plasmaporation.”

As a result of the non-thermal plasma treatment of the inventive process, pores are opened in the subject's skin for enabling transdermal movement of analytes from inside to outside the subject's body. This movement can occur passively in the sense that the skin is left undisturbed (after plasma treatment), with movement of the analytes through the skin being driven by the difference in the concentration levels of this analyte in the fluids underneath and on the surface of the subject's skin. In addition, this movement can also occur actively in the sense that a mechanical device can be used to help drive the analyte from inside to outside of the subject's body. For example, suction can be applied to the subject's skin to help draw the analyte out of the subject's body through the newly formed pores created by the non-thermal plasma treatment of the inventive process.

It will therefore be appreciated that this invention, in its broader aspects, contemplates a process for withdrawing analytes from inside a subject's body noninvasively in which an area of the subject's skin is exposed to a non-thermal plasma, thereby enabling migration of the analyte to the surface of the subject's skin through the newly created temporary pores, and further in which analyte which has migrated to the surface of the skin is recovered for analysis.

Also, for the sake of clarity, it should be understood that when we refer to recovering analytes from “inside the subject's body,” we intend to include analytes which are recovered from inside the subject's skin, underneath its outer surface, as analytes in the subject's skin beneath its outer surface are also “inside the subject's body.”

The details of how non-thermal plasmas can be generated to open pores in a subject's skin are extensively described in the above-mentioned U.S. 2015/0094647. For example, the gases that can be used for generating these non-thermal plasmas include air as well as numerous other gases such as He, Ar, Ne, Xe and the like. Mixtures of inert gases with small percentage (0.5%-20%) of other gases such as O₂ and N₂ can also be used, as can mixtures of inert gases with vaporized liquids including water, DMSO, ethanol, isopropyl alcohol, n-butanol, and other organic solvents, with or without additives and the like. In particular embodiments, He, He+O₂, N₂, He+N₂, Ar, Ar+O₂, Ar+N₂, and the like can be used.

The high voltage power source that can be used for carrying out the reverse plasmaporation step of this invention can be a DC power source, a high frequency AC power source, an RF power source, a pulsed DC power source, a pulsed AC power source, a microwave power source or the like. These high power voltage sources can be operated with a number of different wave forms, such as, for example, a constant, ramp-up, ramp-down, pulsed, nanosecond pulsed, microsecond pulsed, square, sinusoidal, random, in-phase, out-of-phase, and the like. In addition, they can be operated with voltages that range from 3 kV to 30 kV at frequencies ranging from 1 Hz to 20 kHz. They can also be pulsed with a duty cycle of 1-100% and pulse duration of 1 nanosecond up to 1 microsecond. They can also be applied as discrete pulses (1-100 in number) having pulse durations ranging from 1 ns to 10 μs and magnitude of voltage ranging from 3-30 kV.

Desirably, these power sources are operated in a manner which avoids damaging the underlying skin, more desirably in a manner which avoids generating any pain in the subject's skin. One way this can be done is to apply short duration pulses repetitively, which allows the same amount of energy that would otherwise cause damage to be transferred without causing localized heating and skin damage. In any event, it is desirable to operate these high voltage power sources so that the energy deposited on intact skin is less than about 25 J/cm², more typically less than about 10 J/cm², less than about 5 J/cm², or even less than about 3 J/cm².

Particular operating regimens which have been found useful for operating large area DBD plasma generating systems of the type shown in FIG. 1 for generating direct plasmas, as well as for operating large area DBD plasma generating systems of the type shown in FIG. 2 for generating afterglow plasmas, include using applied voltages with pulse widths of between about 40-500 ns (single pulse to 20 kHz) with rise times of 0.5-1 kV/ns and magnitudes of about ˜20 kV (peak-to-peak) at a power densities of 0.01-100 W/cm². Operating regimens using applied voltages having pulse widths between about 1-10 μs (50 Hz to 30 kHz) and magnitudes of about 20 kV (peak-to-peak) are interesting. An operating regimen using a pulse waveform having amplitude of about 22 kV (peak-to-peak), a duration of about 10 μs, frequency of 3500 Hz with a rise time of about 5 V/ns is also of interest, as it generates a discharge power density of about 0.1 W/cm² to 2.08 W/cm². Typical treatment times could range from 1 second to 120 seconds, more typically less than about 90 s, less than 60 s, or even less than about 30 s.

Meanwhile, particular operating regimens which have been found useful for operating a jet-type plasma generating system of the type illustrated in FIG. 3 include using alternating polarity pulsed voltages having pulse widths of between about 1-10 μs at operating frequencies of 50 Hz to 3.5 kHz and duty cycles of 1-100%. Such operating regimens with rise times of 5 V/ns and a magnitude of about ˜20 kV (peak-to-peak) at power densities of 0.1-10 W/cm² are of particular interest.

Finally, filter 230 in the embodiment of FIG. 2 can comprise a copper woven wire cloth having a 16×16 mesh size with a 0.011″ wire diameter and a 0.052″ opening size (67% opening area). However, a mesh made of different conducting materials, wire diameters and opening sizes can also be used.

The surface area of the skin that is treated may be readily increased by scaling up the size of the surface area of the plasma generator or moving the plasma generator along the surface of the skin.

### In Vivo Analytes—Qualitative Analysis

The inventive reverse plasmaporation process can be used to recover a wide variety of different analytes from inside a subject's body. Examples include, in addition to glucose, acetaldehyde, acetate, acetic acid, adenosine 5′-monophosphate, alanine, alcohol ester, aliphatic nitro, compounds, alkaline phosphatase, allyl alcohol, altronolactone, amino acids, aminophenol, ammonia, amp, amylamine, amylase, arginine, aromatic amine, aromatic diamine, arsenate, ascorbic acid, aspartate, benzaldehyde, benzidine, benzylamine, borate, butanol, butylamine, cadaverin, calcium, carbohydrate, catechol, chlorogenic acid, cholesterol, choline, cholinesterase, chymotrypsin, cresol, dextran, dextrose, diamine, dianisidine, dihydro-orotate, dihydroxyacetone, dihydroxyphenylalanine, dioxy-d-glucose, dioxy-fluoro-d-glucose, dopamine, emulsin, erythrose, ethanol, ethyl mercaptan, formaldehyde, formic acid, fructose, furfural, furfuryl alcohol, galactonolactone, galactose, glutamic acid, glucono-lactone, glucopyranose, glucoronidase, glucose-6-phosphate, glucosidase, glutamate, glutamate pyruvate, transaminase, glyceraldehyde, glycerin, glycerol, glycolate, glyoxylate, hexylamine, histamine, histidine, hydrogen peroxide, hydroquinone, hydroxymethyl, furfural, hydroxyphenylacetic acid, hydroxyphenyllactic acid, hypoxanthine, hydroxy acids, inorganic phosphorus, isobutylamine, isopropanol, lactase, lactate, lactate dehydrogenase, lactic acid, lactose, leucine, lipase, lysine, lysine decarboxylase, maltose, mandalate, mannose, mannonolactone, melibiose, methanol, methionine, methyl sulfate, methyl-d-glucose, methyl-l-amino acids, methylcatechol, molybdate, monoamine, monomethyl sulfate, n,n-diethyl-p-phenylenediamine, n,n-dimethyl-p-phenylenediamine, nad, nadh, nadph, nitroethane, octylamine, oxalate, oxalic acid, pectin, pectin esterase, phenol, phenylalanine, phenylenediamine, phosphate, phosphatidyl choline, polyamine, proline, propanol, propylamine, purine, putrescin, pyridoxamine phosphate, pyrocatechol, pyrogallol, pyruvate, pyruvic acid, raffinose, salicin, sarcosine, sorbose, spermidine, spermine, starch, sucrose, sulfite, thiamine, trehalose, tryptophan, tungstate, tyramine, tyrosine, uric acid, valine, verbascose, vitamin b1, vitamin c, xanthine, xylopyranose

It will therefore be appreciated that the inventive process can be used as an effective analytical tool for carrying out qualitative analyses to detect the presence of a wide variety of different analytes in a subject's body.

### Blood Analytes—Quantitative Analysis

In an especially interesting embodiment of this invention, the inventive technology can be used for determining the concentrations of various analytes in a subject's blood.

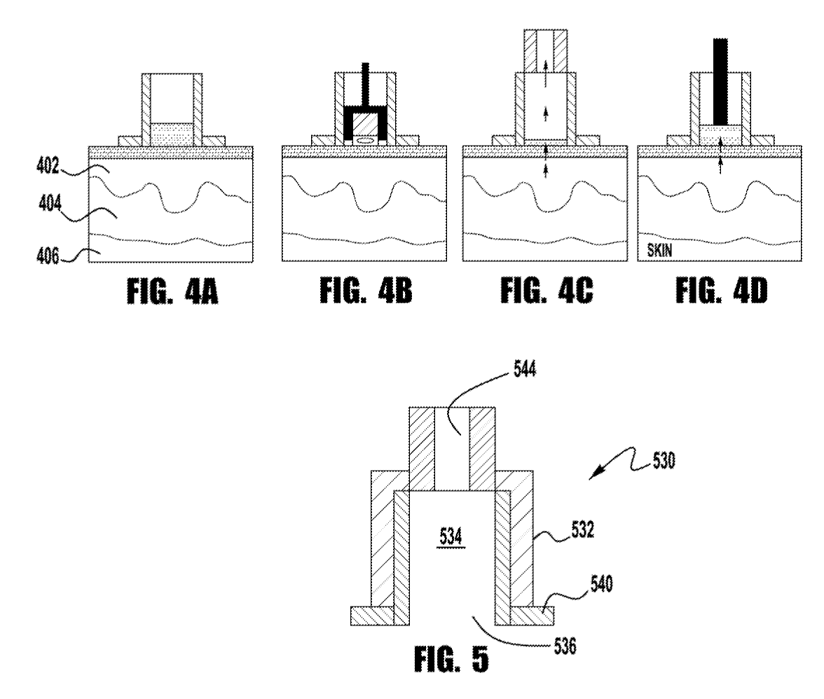

This embodiment is more fully illustrated in FIGS. 4(a), 4(b), 4(c) and 4(d), which are schematic diagrams illustrating the process of this embodiment. For the purpose of illustration, each of these figures show the skin of a subject in expanded form comprising the stratum corneum 402, the epidermis 404 and the dermis 406.

As shown in these figures, this process starts with optional hydration step (a) in which the area of the subject's skin which is to be exposed to a non-thermal plasma is flooded with water or other aqueous fluids. Saline solutions, and especially phosphate-buffered saline, are especially interesting, since they mimic the chemical composition of the interstitial fluids being extracted by the reverse plasmaporation technology of this invention. Hydration times as short as 1 minute or less to as long as 2 hours or more are contemplated, although hydration times on the order of 5 minutes to 1.5 hours, 15 minutes to 1 hour and 30 to 45 minutes are believed to be more likely.

In the next step, step (b), the subject's skin is exposed to non-thermal plasma to increase its permeability by reverse plasmaporation. For this purpose, any of the exemplary non-thermal plasma generating systems described above can be used, as well as other non-thermal plasma generators.

One of the problems associated with prior noninvasive transdermal diagnostics is obtaining sufficient quantities of analyte for detection. One way for dealing with this problem is to use a “large area” dielectric barrier discharge (“DBD”) plasma generating system of the type illustrated in FIGS. 1 and 2 for carrying out the reverse plasmaporation of this step. This is because recovering analyte from a larger area rather than a smaller area results in a greater amount of analyte being recovered per unit time per unit area. Therefore, in those instances in which the speed at which the inventive process can be carried out is an issue, it may be beneficial to use a non-thermal plasma generating system of this type.

In any event, depending on the particular non-thermal plasma generator used as well as the particular conditions at which this generator is operated, step (b) can be as short as 10 seconds and as long as 30 minutes. However, treatment times of 15 seconds to 15 minutes or even 20 seconds to 10 minutes are believed to be more typical. In this regard, it is envisioned that in some embodiments the treatment time will be on the order of 1 to 20, more typically 2 to 10 or even 4 to 8 minutes, while in other embodiments the treatment time will be on the order of 10 to 90 seconds, 15 to 60 seconds or even 20 to 40 seconds. As indicated above, this step is desirably carried out in a manner so as to avoid damaging or otherwise inducing pain in the subject's skin, preferably by limiting exposure of the skin to a maximum energy density of about 25 J/cm².

Next in step (c), analytes existing below the surface of the subject's skin are recovered for analysis. This step can be accomplished by any technique which will recover these analytes in measurable amounts. For example, this can be done in a passive manner such as by (1) allowing the skin which has been subjected to the non-thermal plasma to sit undisturbed for a period of time long enough to enable the analytes to migrate to the skin's surface on their own accord and then (2) recovering these analytes by any suitable technique such as by eluting the skin surface with a suitable carrier liquid or by allowing these analytes to be taken up by a patch of cotton, gauze or other suitable material. These steps (1) and (2) can also be accomplished simultaneously, if desired. This approach is passive in the sense that the driving force which causes an analyte to travel transdermally from inside to outside of the subject's body is the difference in the concentration levels of this analyte in the fluids underneath and on top of the subject's skin.

Step (c) can also be accomplished actively in the sense that a mechanical device can be used to help drive the analyte from inside to outside of the subject's body. For example, one way this can be done is by applying suction to the subject's skin to help draw the analyte out of the subject's body. For this purpose, a vacuum device such as illustrated in FIG. 5 can be used. As shown there, vacuum recovery device 530 comprises a housing 532 defining a vacuum chamber 534 therein including an opening 536 which communicates with the subject's skin when the device is in place. Housing 532 defines extended rim 540 surrounding opening 536, which may include an adhesive backing on its lower surface for helping to create a good seal between rim 540 and the subject's skin when the device is in place. Outlet port 544 communicates with a vacuum device (not shown) for creating a vacuum (i.e., generating a pressure less than the pressure of the surrounding atmosphere) when the device is in use.

In operation, a vacuum is created inside housing 532 by means of the vacuum device connected to outlet port 544. As a result, suction draws the interstitial fluid present in the interstitial space below the surface of skin out of the subject's body through the pores generated in skin during the non-thermal plasma treatment of step (b). Vacuum is maintained for period of time sufficient to recover a measurable amount of this interstitial fluid in housing 532. Normally, this will take about 30 seconds to 10 minutes, more typically 1 minute to 5 minutes, although shorter and longer time periods are possible. Desirably, steps (b) and (c) are carried out using equipment and process parameters which are sufficient so that the amount of fluid collected in housing 532 is at least 0.001 ml, more desirably at least 0.01 ml or at least 0.1 ml.

In another active approach for recovering analytes in this step (c), a microneedle array of the type conventionally used for the delivery of drugs and other substances can be used for recovering this interstitial fluid from underneath the subject's skin. Such microneedle arrays are commercially available and described in many patents, an example of which is U.S. Pat. No. 9,033,898. Basically, these devices comprise an array of extremely small needles and a support structure adapted to advance the distal ends of these needles so that they penetrate only the outermost one or two layers of a subject's skin, i.e., the outermost layer (the stratum corneum) or both the stratum corneum and the layer immediately underneath, the epidermis. As described in the '898 patent and elsewhere, it is already known that such devices can be used for recovering samples of interstitial fluid found underneath the skin.

In accordance with this invention, such devices when adapted for analyte recovery can be used in combination with the reverse plasmaporation technology of this invention to provide an especially effective means for recovering analytes from underneath a subject's skin. This is because the reverse plasmaporation technology of this invention is capable of opening pores in, and thereby substantially increase the permeability of, the last and final layer of the skin, the dermis. Such devices are not capable of doing this on their own. Accordingly, when used together these technologies are capable of achieving an especially effective recovery of analytes from underneath a subject's skin.

On the other hand, using microneedle devices requires that the outer two layers of skin be penetrated, which not only may cause discomfort but also may represent a source of potential infection. So, in those instances in which a truly non-invasive approach for analyte recovery is desired, using microneedle arrays for carrying out step (c) may not be advisable.

As further illustrated in FIGS. 4(a), 4(b), 4(c) and 4(d), the final step in the process of this embodiment, step (d), involves determining the concentration of the analyte in the fluid recovered in step (c) in a manner which enables the concentration of this analyte in the subject's blood to also be determined.

In this regard, please see Kost et al., Transdermal Monitoring of Glucose and Other Analytes Using Ultrasound, NATURE MEDICINE, vol. 6, number 5, pp 347-350, March 2000, © 2000 Nature America, http://medicine.nature.com, which describes a process for recovering analytes from underneath a subject's skin using ultrasound as the means for enhancing the amount of analytes recovered. As described there, the amount of glucose and other analytes recovered from the interstitial fluid obtained from underneath the skin, when measured in terms of analyte flux, bears a remarkable similarity to the concentration of this analyte in the subject's blood. See, especially, FIG. 2(a) of this publication. This, in turn, enables a direct correlation to be made between the concentration of the analyte in the recovered analyte fluid and the concentration of this analyte in the subject's blood, requiring only a single (i.e., one point) calibration between a measured transdermal analyte flux and a measured blood analyte concentration. See, especially, FIG. 2(b) and page 350 of this publication.

Accordingly, this same approach can be used in step (d) of the process of this embodiment for accurately predicting the concentration of the analyte in a subject's blood based on the measured amount of analyte recovered by the process of this embodiment. In particular, based on the amount of analyte contained in the sample analyte fluid recovered in step (c) as well as the conditions of this recovery process including the area of the skin from which this fluid was recovered as well as the time over which this recovery occurred, the rate at which this analyte was recovered per unit area, i.e., the analyte flux, can be calculated. From this relationship, and based on just a single calibration between a measured transdermal analyte flux and a measured blood analyte concentration, the relationship between the concentration of the analyte in the recovered analyte fluid and the concentration of this analyte in the subject's blood can be determined.

In order to more thoroughly describe this invention, the following working examples are provided. In these examples, the ability of the inventive reverse plasmaporation process to aid in the detection of caffeine in a human subject was demonstrated in the following manner.

### EXAMPLE 1

Freshly preserved frozen human skin, obtained from Science Care, was thawed, cut in to sample pieces measuring 1 in.×1 in. (˜2.54 cm×˜2.54 cm), and then mounted in a modified Franz Cell Chamber. A Franz Cell Chamber is an analytical device used to measure the diffusion of drugs through skin. For these experiments, a modified Franz Cell Chamber was used which was structured to enable the surface of the skin samples being tested to be plasma treated. For this purpose, the donor chamber of the device for containing the test fluid to be analyzed was arranged below the skin sample being tested while the receptor chamber of the device for receiving migrated analytes was arranged above the skin sample and structured to receive the electrode of a cold plasma generator.

To carry out each analysis, an aqueous test solution of caffeine in a concentration of 100 microgram/ml was deposited in the donor chamber of the device and the skin sample to be tested then mounted in the device with its lower surface in intimate contact with the aqueous test solution. Thereafter, the skin sample was allowed to equilibrate for 1 hour to allow the skin sample to begin up-taking caffeine through natural skin migration, thereby mimicking conditions in a patient's skin. During this 1 hour equilibration period, the upper surface of the skin was bathed in a physiological buffer (phosphate buffered saline) comprising sodium chloride (8 g/L), potassium chloride (0.2 g/L), sodium phosphate dibasic (1.42 g/L) and potassium phosphate (0.24 g/L).

After termination of the equilibration period, the buffer was removed and the upper surface of the skin was subjected to a non-thermal DBD (dielectric barrier discharge) cold plasma treatment under the following conditions: 17 kV, 2500 Hz, 5 microsecond pulse duration and 100% duty cycle.

Immediately after this cold plasma treatment, the upper surface of the skin was bathed again in the physiological buffer. Thereafter, at 0 minutes, 15 minutes, 60 minutes and 120 minutes after the buffer was applied, 200 microliter samples of the fluid on the skin surface were recovered and analyzed by High Performance Liquid Chromatography to determine the concentration of caffeine in each sample.

Two runs as described above were conducted, one in which the plasma treatment lasted 30 seconds and the other in which the plasma treatment lasted 60 seconds. In addition, a third control run was also carried out in the same way as described above, except that the plasma treatment was omitted.

The results obtained are illustrated in FIG. 6. As shown there, at time zero, i.e., immediately after the plasma treatment had ended and the buffer applied, no caffeine was discerned in all three cases. This shows that, in all of these cases, the skin samples had not been in contact with the caffeine containing test solution long enough to enable caffeine molecules to migrate to the skin surface though naturally occurring skin migration. And this is so, whether or not the skin had been subjected to plasma treatment.

However, at the 15 minute mark, all three interstitial fluid samples showed some migration of caffeine to the skin surface. Interestingly, at this time, the amount of caffeine that had migrated to the surface of the skin that had been plasma treated for 60 seconds was less than the amount that migrated to the surface of the untreated skin. In contrast, the amount of caffeine that had migrated to the surface of the skin that had been plasma treated for 30 seconds was more than the amount that migrated to the surface of the untreated skin.

FIG. 6 further shows that, at the 60 minute mark, the amount of caffeine that had migrated to the surface of the skin that had been plasma treated for 30 seconds was still more than the amount of caffeine that had migrated to the surface of the untreated skin. However, unlike the results at the 15 minute mark, at the 60 minute mark FIG. 6 also shows that the amount of caffeine that had migrated to the surface of the skin that had been plasma treated for 60 seconds was just about the same as the amount of caffeine that had migrated to the surface of the untreated skin.

FIG. 6 further shows that, at the 120 minute mark, the amount of caffeine that had migrated to the surface of the plasma treated skin was more than the amount of caffeine that had migrated to the surface of the untreated skin for both plasma treatments. More importantly, FIG. 6 still further shows that the greatest amount of caffeine migration occurred at the 120 minute mark in the skin sample subjected to the most plasma, i.e., the 60 second plasma treatment.

Together, these results demonstrate that treating the skin with a cold plasma in accordance with the inventive reverse plasmaporation process does, indeed, facilitate the migration and non-invasive recovery of analytes existing underneath the surface of the skin. This, it is believed, should enable reasonable estimates of the concentrations of these analytes in a subject's blood to be made based on the types and amounts of these analytes found.

### EXAMPLE 2

Example 1 was repeated, except that hydration of the skin samples with a physiological buffer before plasma treatment was omitted.

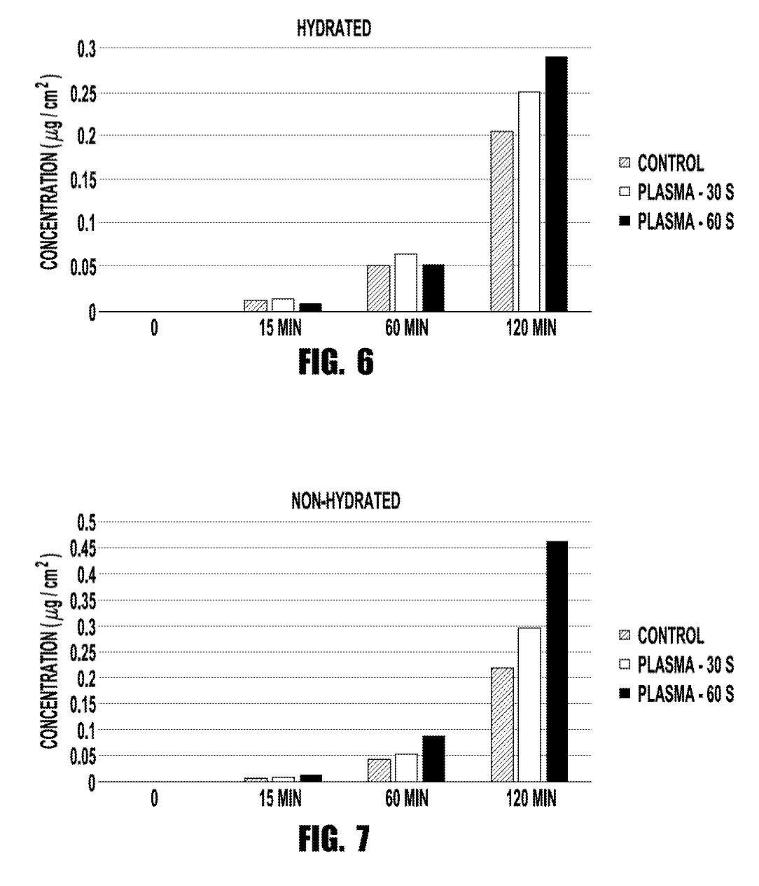

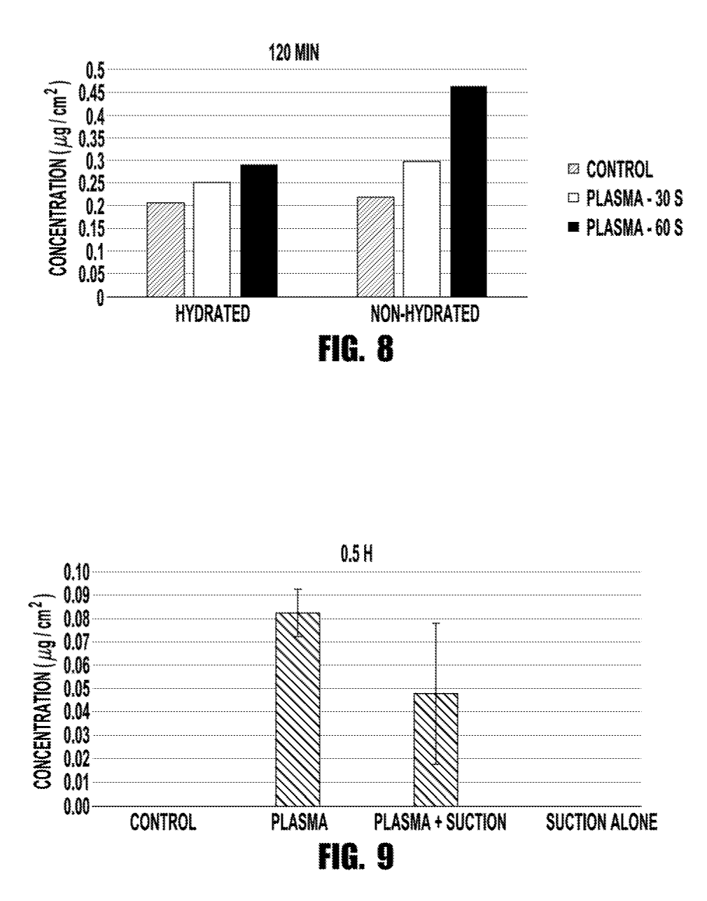

The results obtained are illustrated in FIG. 7, while a comparison of the results obtained in both Examples 1 and 2 at the 120 minute mark are illustrated in FIG. 8.

FIG. 7 shows that, as in the case of Example 1, at the 120 minute mark, both plasma treated skin samples exhibited a greater amount of caffeine migration and recovery than the untreated skin. However, unlike the case of Example 1, in the case of Example 2, FIG. 7 also shows that both plasma treated skin samples exhibited a greater amount of caffeine migration than the untreated skin at the 60 minute mark and the 15 minute mark as well. In addition, FIG. 7 further shows that the amount of caffeine recovery for both plasma treated skin samples, and especially the skin sample plasma treated for 60 seconds, far exceeded the amount caffeine recovered by the untreated skin.

Finally, FIG. 8, which is a comparison of the results obtained in both Examples 1 and 2 at the 120 minute mark, shows that the absolute amount of caffeine recovered from both plasma treated skin samples in Example 2, and especially the skin sample plasma treated for 60 seconds, was substantially more than that recovered in Example 1. This suggests that faster results can be obtained using the inventive reverse plasmaporation technology if a skin hydration with a physiological buffer is omitted.

### EXAMPLE 3

Example 1 was repeated, except that in this example, porcine skin was used. Porcine skin is thicker than human skin and in addition, a suction device such as illustrated in FIG. 5 operating at 150 mm Hg for 2 minutes was used to recover the samples from the surface of the skin. Moreover, to assess the benefit of using suction in addition, three different sets of control runs were also carried out for comparison purposes. In the first set of control runs, no plasma or suction was used to aid analyte recovery. In the second set of control runs, only plasma was used to aid plasma recovery. In the third set of control runs, only suction was used to aid analyte recovery.

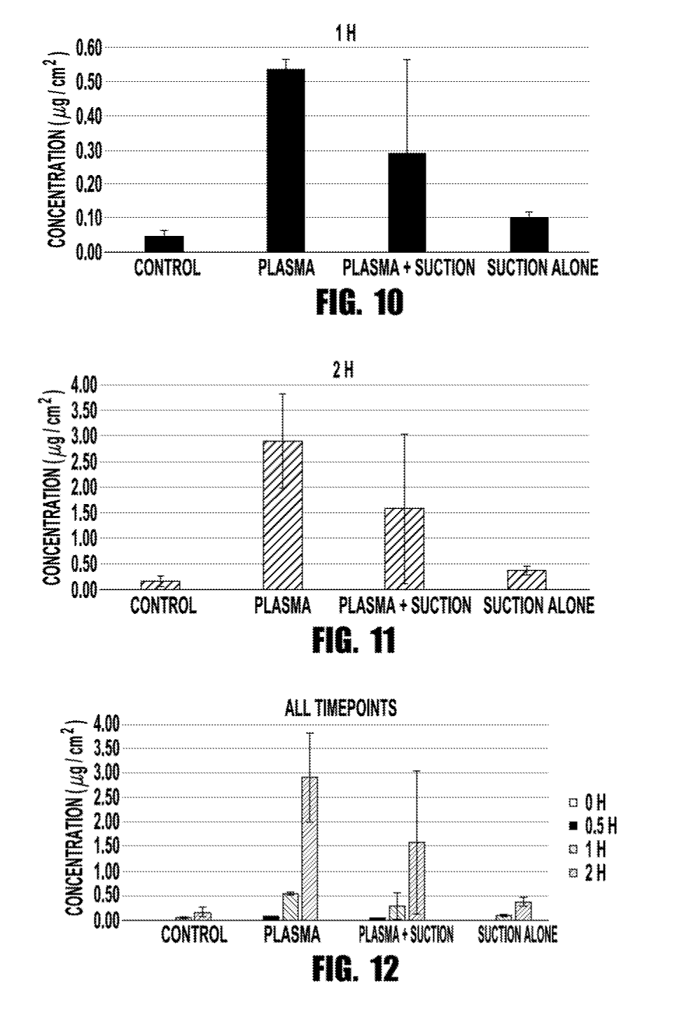

The results obtained are illustrated in FIGS. 9-12 in which FIGS. 9, 10 and 11 are bar charts recording the results obtained at the 30, 60 and 120 minute marks, respectively, while FIG. 12 is a bar chart showing all of these results together. Note that, in FIG. 12, for each separate mode of operation (i.e., no plasma or suction, plasma alone, suction alone and plasma plus suction) the data recovered at all four different measurement times (i.e., at time zero, 30 minutes, 60 minutes and 120 minutes) is set forth in FIG. 12. This means that, where no vertical bar is visible for representing the results obtained for a particular mode of operation at a particular time point, the results obtained were too small to be measured.

As shown in these figures, at each time point starting at the 30 minute mark, the best performance was exhibited by the samples treated with plasma only. This demonstrates that using suction to assist recovering of analytes by reverse plasmaporation not only may be unnecessary but, indeed, may also be counterproductive at least in some instances.

Although only a few embodiments of this invention have been described above, it should be appreciated that many modifications can be made without departing from the spirit and scope of this invention. For example, although the above disclosure indicates that different mechanical devices are used to carry out each of steps (a), (b), (c) and (d) of the process of FIG. 4, it should be appreciated that the hardware used to carry out each of these steps can be combined together in one or more combination devices. For example, the hydration function of step (a) and the non-thermal plasma generation function of step (b) can be combined in the same device.

Alternatively, the non-thermal plasma generation function of step (b) can be combined in the same device with a vacuum assembly for accomplishing the analyte recovery function of step (c). In addition, such a device could be provided with window for enabling access of a probe to be inserted in to the collected analyte fluid for detecting, measuring and quantifying the analyte. Alternatively, the analyte recovery function of step (c) and the analysis function of step (d) could by combined in a single device such as a microneedle patch equipped with a suitable analyzer and computer programmed to automatically determine the concentration of the analyte in the subject's blood based on the results of the analysis carried out.

Finally, all four functions, i.e., hydration, non-thermal plasma generation, analyte recovery and analyte analysis could be combined in the same device.

Still other modifications that can be made include using chemical permeation enhancers before or after plasma treatment for promoting more efficient pore formation, formation of more pores per unit area and for lengthening the time the pores remain open. Examples of suitable compounds that can be used for this purpose include surfactants such as sodium lauryl sulfate, sodium dodecyl sulfate, polyethoxylated sorbitan esters and sorbitan esters, fatty acids such as oleic acid and lauric acid, azones, ethanol, DMSO and isopropyl myristate. In addition, a cloth or gauze patch could be used to recover the extracted analytes, which could then be removed for remote analysis.

In addition to or as an alternative of using chemical permeation enhancers, still another approach for promoting more efficient pore formation and analyte permeation is to use one or more strips of adhesive tape to remove a few layers of the stratum corneal before applying the plasma. The adhesive tape does not hurt or cause irritation but rather merely reduces the thickness of the stratum corneal. Normally, this optional adhesive tape step would occur before the optional hydration step, although this is not absolutely necessary.

Still another modification that can be made is to use the open pores created by the reverse plasmaporation of step (b) for administering drugs and other molecules indicated by the analysis obtained in step (d). One way this could be done automatically would be to include, in a combination device adapted to carry out the reverse plasmaporation function of step (b) as well as the analyte recovery and analyte analysis functions of steps (c) and (d), structure including a diagnostic protocol for determining the type and/or amount of drug that should be delivered as well as a delivery system for delivering this drug to the surface of the subject's skin. For example, the device could include a reservoir of a drug, for example insulin, as well as a feedback loop which would automatically direct the device to administer a predetermined amount of this drug in response to the analysis obtained in step (d). Such a device would first extract the analyte, then determine the concentration of the analyte in the subject's blood, compare this value to a normal range in a healthy subject, determine the amount of drug that would be needed to be administered to return the concentration of this analyte in the subject's blood to normal range, and finally direct the device to administer this amount of the drug to the subject before the pores in its skin created by the non-thermal plasma close. Alternatively, the device could administer this amount of drug the subject after the pores close, either using a separate, later step to reopen the pores and/or administering the drug in another way.

Yet another modification relates to the use of the reverse plasmaporation technology of this invention for purposes other than recovering analytes for analysis. In this regard, because this technology facilitates the removal of substances from inside a subject's body, this technology can be used in other applications where such removal is desired. For example, this technology can be used for facilitating liposuction, collection of cerebrospinal fluid (CSF), collection of synovial fluid from joints, the treatment of cysts, and so forth.

All such modifications are intended to be included within the scope of this invention, which is to be limited only by the following claims:

1. A process for recovering an analyte from inside a subject's body comprising exposing an area of the subject's skin to a non-thermal plasma, thereby enabling migration of the analyte from inside the subject's body to the surface of the subject's skin, and then recovering the analyte from this surface.

2. The process of claim 1, wherein the non-thermal plasma is one of a DBD plasma and a DBD plasma jet.

3. The process of claim 2, wherein the non-thermal plasma is a DBD plasma structured to apply a direct plasma to the subject's skin.

4. The process of claim 2, wherein the non-thermal plasma is a DBD plasma structured to apply an afterglow plasma to the subject's skin.

5. The process of claim 2, wherein the non-thermal plasma is a DBD plasma jet.

6. The process of claim 1, wherein the analyte is recovered from the surface of the subject's skin.

7. The process of claim 6, further comprising analyzing the fluid to determine analyte flux.

8. The process of claim 6, further comprising analyzing the fluid to determine the concentration of the analyte in the subject's blood.

9. The process of claim 1, further comprising hydrating the surface of the subject's skin to be exposed to the non-thermal plasma by contacting this skin with an aqueous liquid.

10. The process of claim 9, wherein the aqueous liquid is phosphate-buffered saline.

11. The process of claim 1, wherein the analyte is recovered from the surface of the subject's skin by means of microneedles.

12. The process claim 1, further comprising hydrating the surface of the subject's skin to be exposed to the non-thermal plasma by contacting this skin with an aqueous liquid.

13. The process of claim 12, wherein the aqueous liquid is a phosphate-buffered saline.

14. The process of claim 1, further comprising contacting the area of the subject's skin with a chemical permeation enhancer before exposing this area of skin to the non-thermal plasma.

15. A process for determining the concentration of an analyte in a subject's blood comprising exposing an area of the subject's skin to a non-thermal plasma, thereby enabling migration of the analyte to the surface of the subject's skin, recovering the analyte from the surface of the subject's skin, and then determining the concentration of this analyte in the subject's blood based on the amount of analyte recovered from the surface of the subject's skin.

16. The process of claim 15, wherein the non-thermal plasma is a direct DBD plasma.

17. The process of claim 15, further comprising determining the concentration of the analyte in the subject's blood by determining the concentration of the analyte in the recovered fluid.

18. The process of claim 15, further comprising hydrating the surface of the subject's skin to be exposed to the non-thermal plasma by contacting this skin with an aqueous liquid.

19. A device for automatically maintaining the concentration of a blood analyte found in a subject's blood to within a desired predetermined range comprising a plasma generator arranged to expose the subject's skin to a non-thermal plasma thereby enabling migration of a quantity of this blood analyte to the surface of the subject's skin, an analytical device for measuring this quantity of blood analyte, a dispenser for delivering a quantity of a drug to the subject, and a control system for directing the dispenser to administer a predetermined amount of this drug to the subject in response to the amount of this blood analyte measured by the analytical device.

20. The device of claim 19, wherein the dispenser is arranged to deliver the predetermined amount of this drug to the subject by applying this drug to the same area of the subject's skin which has been exposed to the non-thermal plasma.
